(19) United States
(12) Patent Application Publication
(10) Pub. No.: US 2022/0105499 A1
(43) Pub. Date: Apr. 7, 2022

(54) COMBINED CATALYST AND PREPARATION METHOD THEREOF, AND METHOD FOR PREPARING XYLENE BY COUPLING CARBON DIOXIDE HYDROGENATION WITH TOLUENE ALKYLATION

(71) Applicant: XIAMEN UNIVERSITY, Xiamen, Fujian (CN)

(72) Inventors: Youzhu YUAN, Xiamen, Fujian (CN); Jiachang ZUO, Xiamen, Fujian (CN); Xinping DUAN, Xiamen, Fujian (CN); Linmin YE, Xiamen, Fujian (CN); Xuelian LIANG, Xiamen, Fujian (CN); Haiqiang LIN, Xiamen, Fujian (CN)

(21) Appl. No.: 17/261,638

(22) PCT Filed: Mar. 2, 2020
(86) PCT No.: PCT/CN2020/077412

§ 371 Date: Jan. 20, 2021

(30) Foreign Application Priority Data
Nov. 21, 2019 (CN) 201911149539.2

(51) Int. Cl. B01J 29/40; B01J 35/00; B01J 23/06; B01J 37/04; B01J 37/08; B01J 37/03; B01J 37/18; C07C 2/86

(52) U.S. Cl. CPC B01J 29/405; B01J 35/0006; B01J 23/06; B01J 37/04; B01J 37/082; C07C 2523/06; B01J 37/18; C07C 2/862; B01J 2229/20; C07C 2529/40; B01J 37/031

(57) ABSTRACT

The technical field of catalysts, in particular to a combined catalyst and a preparation method thereof, and a method for preparing xylene by coupling carbon dioxide hydrogenation with toluene alkylation. The combined catalyst of the present disclosure having a metal oxide and a zeolite. In the present disclosure, the metal oxide is mainly used to reduce carbon dioxide to methanol, and the zeolite is mainly used to react toluene with methanol to produce xylene. When the catalyst of the present disclosure is used to prepare xylene, carbon dioxide and hydrogen can be used as raw materials instead of methanol. Compared with the traditional alkylation of toluene with methanol, this method can avoid the side reaction of methanol to olefins caused by the improper methanol/toluene feeding ratio, and improve the production efficiency of xylene; meanwhile, it can inhibit xylene isomerization and increase p-xylene selectivity in the products.

The application claims the priority of Chinese Patent Application No. CN201911149539.2, entitled “Combined catalyst and preparation method thereof, and method for preparing xylene by coupling carbon dioxide hydrogenation with toluene alkylation” filed with the China National Intellectual Property Administration on Nov. 21, 2019, which is incorporated herein by reference in its entirety.

### FIELD OF THE INVENTION

The present disclosure relates to a technical field of catalysts, and particularly to a combined catalyst and a preparation method thereof, and a method for preparing xylene by coupling carbon dioxide hydrogenation with toluene alkylation.

### BACKGROUND OF THE INVENTION

At present, the industrial methods for preparing xylene mainly include toluene disproportionation, transalkylation of toluene with trimethylbenzene, and alkylation of toluene with methanol. Among them, alkylation of toluene with methanol involves an environmentally friendly reaction, and the resulting theoretical by-product is only water; the alkylation of toluene with methanol is an electrophilic substitution reaction that occurs at Brønsted acid sites. It is generally believed that methanol is first dehydrogenated over the zeolite to generate methoxy group, and then the methoxy group attacks H atom on toluene to complete the substituted alkylation reaction, while releasing the acidic protons over the zeolite.

Generally, zeolite is used as the catalyst for the alkylation of toluene with methanol to produce xylene, but the zeolite catalyst has too many acidic sites, which lead to xylene isomerization, and thus the obtained products show thermodynamic distribution state, and a low selectivity to p-xylene (hereinafter referred as PX), thereby leading to a higher energy consumption for separating PX, m-xylene, and o-xylene.

### SUMMARY OF THE INVENTION

An object of embodiments of the present disclosure is to provide a combined catalyst, and when used to prepare PX, the catalyst can inhibit the xylene isomerization and improve PX selectivity in products.

In order to achieve the above-mentioned object, the present disclosure provides the following technical solutions:

The present disclosure provides a combined catalyst, comprising a metal oxide and a zeolite; the metal oxide includes one or more of ZnZrOₓ₁, ZnCrOₓ₂, ZnAlOₓ₃ and CrOₓ₄, wherein 1<x1<2, 1<x2<1.5, 1<x3<1.5, and 1<x4<1.5.

In some embodiments, the zeolite includes one or more of ZSM-5 zeolite, MCM 22 zeolite, and SAPO-34 zeolite.

In some embodiments, a mass ratio of the metal oxide to the zeolite is (1-9):(1-9).

The present disclosure provides a method for preparing the combined catalyst as described in the above technical solutions, comprising:

mixing the metal oxide with the zeolite, to obtain the combined catalyst.

The present disclosure provides another method for preparing the combined catalyst as described in the above technical solutions, comprising:

subjecting a metal salt solution and the zeolite to a first mixing, to obtain a dispersion liquid;

subjecting the dispersion liquid and a precipitant to a second mixing and subjecting the resulting mixture to a precipitation reaction, to obtain a precipitate; and

calcining the precipitate, to obtain the combined catalyst.

The present disclosure also provides a method for preparing xylene by coupling carbon dioxide hydrogenation with toluene alkylation, comprising:

placing the combined catalyst as described in the above technical solutions in a reducing atmosphere, and subjecting to an activation, to obtain an activated catalyst; and

contacting the activated catalyst with carbon dioxide, hydrogen and toluene and subjecting to a carbon dioxide hydrogenation coupled toluene alkylation reaction, to obtain xylene.

In some embodiments, a gas for providing the reducing atmosphere is a mixture of hydrogen and argon, or a mixture of hydrogen and nitrogen.

In some embodiments, the activation is performed at a temperature of 200-600° C. for 0.5-12 hours.

In some embodiments, the space velocity of carbon dioxide is 300-6000 mL·g⁻¹·h⁻¹; a volume ratio of carbon dioxide to hydrogen is 1:(1-8); a molar ratio of carbon dioxide to toluene is (1-30):2.

In some embodiments, the carbon dioxide hydrogenation coupled toluene alkylation reaction is carried out at a temperature of 300-460° C., and a pressure of 1-5 MPa.

The present disclosure provides a combined catalyst, comprising a metal oxide and a zeolite; the metal oxide includes one or more of ZnZrOₓ₁, ZnCrOₓ₂, ZnAlOₓ₃ and CrOₓ₄, wherein 1<x1<2, 1<x2<1.5, 1<x3<1.5, and 1<x4<1.5. In the present disclosure, the metal oxide is mainly used to reduce carbon dioxide to methanol, and the zeolite is mainly used to react toluene with methanol to produce xylene. When the catalyst provided by the present disclosure is used to prepare xylene, carbon dioxide and hydrogen can be used as raw materials instead of methanol. Compared with the traditional alkylation of toluene with methanol, this method can avoid the side reaction of methanol-to-olefins caused by the improper methanol/toluene feeding ratio, and improve the production efficiency of xylene; meanwhile, it can inhibit xylene isomerization reaction and increase the selectivity of PX in products.

The present disclosure also provides a method for preparing xylene by coupling carbon dioxide hydrogenation with toluene alkylation. In the present disclosure, carbon dioxide and hydrogen instead of conventional methanol are used, carbon dioxide is hydrogenated to produce methanol, and the methanol is then subjected to an alkylation reaction with toluene. The consumption of methanol by the alkylation reaction improves the conversion of the carbon dioxide hydrogenation reaction. The alkylation reaction “takes on demand” the methanol generated by the carbon dioxide hydrogenation reaction, preventing the methanol-to-olefins reaction caused by an excessively high methanol concentration, and thus it is beneficial for increasing the yield of xylene, and can slow down the deactivation of the catalyst caused by carbon deposition.

### BRIEF DESCRIPTION OF THE DRAWINGS

FIG. 1 shows a schematic diagram of the instrument used to evaluate and analyze the performance of the combined catalyst; where 1 represents a steel cylinder, 2 represents a pressure reducing valve, 3 represents a three-way valve, 4 represents a pressure regulating valve, 5 represents a pressure gauge, 6 represents a temperature controller, 7 represents a high-pressure injection pump, 8 represents a steel pipe, 9 represents a quartz reaction tube, 10 represents a heating furnace, and 11 represents a condenser.

FIG. 2 is a graph showing 100-hour stability test results of the combined catalyst as prepared in Example 4.

### DETAILED DESCRIPTION OF THE INVENTION

The present disclosure will be further described below in reference to embodiments and drawings.

The present disclosure provides a combined catalyst, comprising a metal oxide and a zeolite; the metal oxide includes one or more of ZnZrOₓ₁, ZnCrOₓ₂, ZnAlOₓ₃ and CrOₓ₄, wherein 1<x1<2, 1<x2<1.5, 1<x3<1.5, and 1<x4<1.5.

The combined catalyst provided in the present disclosure comprises a metal oxide; the metal oxide includes one or more of ZnZrOₓ₁, ZnCrOₓ₂, ZnAlOₓ₃ and CrOₓ₄, wherein 1<x1<2, 1<x2<1.5, 1<x3<1.5, and 1<x4<1.5. In some embodiments of the present disclosure, the metal oxide is prepared by a coprecipitation method. The coprecipitation method for preparing the metal oxide is specified as follows: mixing the corresponding metal salt with a precipitant, and subjecting to a coprecipitation reaction, to obtain a precipitate; subjecting the precipitate in sequence to a washing and a calcining, to obtain the metal oxide. In some embodiments of the present disclosure, the metal salt is one or more of metal nitrate, metal acetate, and metal sulfate. In some embodiments, the precipitant is one or more of ammonia water, ammonium carbonate, sodium hydroxide, potassium hydroxide, sodium carbonate, potassium carbonate, sodium bicarbonate, and potassium bicarbonate. In some embodiments, the washing is performed for 1-5 times. In some embodiments, the detergent used in the washing is deionized water and/or ultrapure water. In some embodiments, the calcining is performed in air. In some embodiments, the calcining is performed at a temperature of 400-700° C. In some embodiments, the calcining is performed for 2-12 hours.

In the present disclosure, the combined catalyst also comprises a zeolite, wherein the zeolite preferably includes one or more of ZSM-5 zeolite, MCM-22 zeolite, and SAPO-34 zeolite, for example H-ZSM-5 zeolite. In some embodiments of the present disclosure, the zeolite is a modified zeolite, for example a tetraethyl orthosilicate-modified zeolite.

In some embodiments of the present disclosure, a method for preparing the modified zeolite is specified as follows: mixing a zeolite with tetraethyl orthosilicate, and subjecting to an impregnation and a calcining, to obtain the modified zeolite. In some embodiments of the present disclosure, a mass ratio of the zeolite to tetraethyl orthosilicate is 1:(0.5-2), for example 1:1. In some embodiments of the present disclosure, the mixing is preformed in a solvent. In some embodiments of the present disclosure, a mass ratio of the zeolite to the solvent is (1-5):1, for example 2.5:1. In some embodiments, the solvent is selected from the group consisting of hexane, pentane, heptane, octane, N,N-dimethylformamide, and N,N-dimethylacetamide. In some embodiments of the present disclosure, the impregnation is preformed for 1-24 hours, for example 4 hours. In some embodiments, the calcining is preformed at a temperature of 400-700° C. In some embodiments, the calcining is preformed for 1-12 hours. In some embodiments, the calcining is preformed in an oxygen containing atmosphere. In some embodiments, the gas for providing the oxygen containing atmosphere is air, oxygen, a mixture of nitrogen and oxygen, a mixture of argon and oxygen, or a mixture of helium and oxygen.

In the present disclosure, in order to ensure fully blocking the framework acidic sites on the external surface of the zeolite, the modification steps as described above could be repeated for several times, generally for 1-8 times. In present disclosure, the modification to the zeolite framework could block the acidic sites on the external surface, limit xylene isomerization, and is beneficial for improving PX selectivity in products.

In some embodiments of the present disclosure, a mass ratio of the metal oxide to the zeolite is (1-9):(1-9), for example 1:(1-9). The present disclosure could effectively improve the xylene selectivity and suppress the reversed water-gas shift reaction by regulating the mass ratio of the metal oxide to the zeolite.

The present disclosure provides a method for preparing the combined catalyst as described in the above technical solutions, comprising: mixing the metal oxide with the zeolite, to obtain the combined catalyst.

In some embodiments of the present disclosure, the means for mixing includes grinding, ball milling, impregnation, precipitation deposition, solvothermal, coprecipitation, or molten salt mixing, and for example grinding or ball milling. In the present disclosure, the metal oxide and the zeolite are fully contacted by mixing, thereby improving the mass transfer effect. When preparing xylene, the coupling of the hydrogenation of carbon dioxide to methanol and the alkylation reaction of toluene with methanol can be realized through migration and conversion of reaction intermediate species.

In some embodiments of the present disclosure, after the mixing is completed, the obtained mixture is granulated and sieved to obtain a combined catalyst.

The present disclosure provides another method for preparing the combined catalyst, comprising:

subjecting a metal salt solution and the zeolite to a first mixing, to obtain a dispersion liquid;

subjecting the dispersion liquid and a precipitant to a second mixing and subjecting the resulting mixture to a precipitation reaction, to obtain a precipitate; and

calcining the precipitate, to obtain the combined catalyst.

In the present disclosure, the metal salt solution and the zeolite are subjected to a first mixing, to obtain a dispersion liquid. In some embodiments of the present disclosure, the metal salt solution is one or more of metal nitrate solution, metal acetate solution, and metal sulfate solution, for example a mixed solution of zinc nitrate and zirconium nitrate. In some embodiments of the present disclosure, the solvent of the metal salt solution is water. In some embodiments of the present disclosure, the concentration of the metal salt solution is 0.010-0.200 g·mL⁻¹, for example 0.065 g·mL⁻¹. In specific embodiments of the present disclosure, under the condition that the metal oxide is ZnZrOₓ₁ (1<x1<2), the in-situ precipitation method is used.

In some embodiments of the present disclosure, a mass ratio of the metal salt in the metal salt solution to the zeolite is (25-35):(65-75), for example 28:72.

In the present disclosure, the specific types of the zeolite and the preparation method of the modified zeolite are the same as those described above, and will not be repeated here.

The present disclosure has no particular limitation on the specific means for the first mixing, and it is advisable to uniformly disperse the zeolite in the metal salt solution.

According to the present disclosure, after the dispersion liquid is obtained, the dispersion liquid and a precipitant is subjected to a second mixing and the resulting mixture are subjected to a precipitation reaction, to obtain a precipitate. In some embodiments of the present disclosure, the precipitant is one or more of ammonia, ammonium carbonate, sodium hydroxide, potassium hydroxide, sodium carbonate, potassium carbonate, sodium bicarbonate, and potassium bicarbonate; in some embodiments, the precipitant is in the form of an aqueous solution when mixed with the dispersion liquid. In some embodiments, the aqueous solution of the precipitant is an aqueous solution of ammonium carbonate; in some embodiments, the concentration of the aqueous solution of the precipitant is 0.02-0.04 g·mL⁻¹, for example 0.031 g·mL⁻¹. In some embodiments of the present disclosure, a mass ratio of the precipitant to the zeolite is (10-20):(80-90), for example 15:85. In some embodiments of the present disclosure, the second mixing is performed at a temperature of 60-80° C., for example 70° C.; in some embodiments, the method for the second mixing is specified as follows: dropwise adding an aqueous solution of the precipitant to the dispersion liquid, in some embodiments at a speed of 1-5 mL·min⁻¹, and for example 3 mL·min⁻¹.

In some embodiments of the present disclosure, the precipitation reaction is carried out at a temperature of 60-80° C., for example 70° C.; in some embodiments, the precipitation reaction is carried out for 1-3 hours, for example 2 hours; the timing is started when the adding of the precipitant is completed. During the precipitation reaction process in the present disclosure, the metal salt reacts with the precipitant to precipitate on the surface of the zeolite in situ, which simplifies the preparation process while ensuring full contact between the metal oxide and the zeolite.

In some embodiments of the present disclosure, the resulting system after precipitation reaction is subjected to a solid-liquid separation, to obtain a precipitate. In some embodiments of the present disclosure, the solid-liquid separation is performed by a centrifugation.

In some embodiments of the present disclosure, the precipitate after centrifugation is subjected to several washings and solid-liquid separations, to remove excess ions. The detergent used in the washing is deionized water, distilled water, or ultrapure water. In some embodiments, the amount of the detergent used in each washing is 3-5 times the volume of the original mixed solution, and the number of the washing and solid-liquid separation is 3-5 times.

According to the present disclosure, after obtaining the above-mentioned washed precipitate, the precipitate is calcined to obtain a combined catalyst. In some embodiments of the present disclosure, the calcination is performed in air; in some embodiments, the calcination is performed at a temperature of 400-700° C.; in some embodiments, the calcination is performed for 2-12 hours.

According to the present disclosure, in some embodiments, the precipitate is dried before the calcination; in some embodiments, the drying is performed at a temperature of 80-120° C., for example 105° C.; in some embodiments, the drying is performed for 8-16 hours.

According to some embodiments of the present disclosure, after calcination, the calcined product is granulated and sieved, to obtain a combined catalyst.

In some embodiments of the present disclosure, the combined catalyst has a particle size of 40-60 mesh. Through granulation, the present disclosure can eliminate the influence of internal diffusion rate on the intrinsic performance of the catalyst.

The present disclosure further provides a method for preparing xylene by coupling carbon dioxide hydrogenation with toluene alkylation, comprising:

placing the combined catalyst as described in the above technical solutions in a reducing atmosphere, and subjecting to an activation, to obtain an activated catalyst; and contacting the activated catalyst with carbon dioxide, hydrogen and toluene, and subjecting to a carbon dioxide hydrogenation coupled toluene alkylation reaction, to obtain xylene.

In the present disclosure, the combined catalyst is placed in a reducing atmosphere and subjected to an activation, to obtain an activated catalyst. In some embodiments of the present disclosure, a gas for providing the reducing atmosphere is a mixture of hydrogen and argon, or a mixture of hydrogen and nitrogen; in some embodiments, under the condition that the gas for providing the reducing atmosphere is a mixture of hydrogen and argon, a volume ratio of hydrogen to argon is 5:95; in other embodiments, under the condition that the gas for providing the reducing atmosphere is a mixture of hydrogen and nitrogen, a volume ratio of hydrogen to nitrogen is 5:95.

In some embodiments of the present disclosure, the activation is performed at a temperature of 200-600° C., for example 450° C.; in some embodiments the activation is performed for 0.5-12 hours, for example 2 hours. In the present disclosure, the activation is to make the catalyst work as soon as possible, and to improve the catalytic reaction abilities of hydrogen, carbon dioxide, and toluene.

According to the present disclosure, after the activated catalyst is obtained, the activated catalyst is contacted with carbon dioxide, hydrogen, and toluene, and the resulting mixture is subjected to a carbon dioxide hydrogenation coupled toluene alkylation reaction, to obtain xylene. In some embodiments of the present disclosure, the space velocity of carbon dioxide is 300-6000 mL·g⁻¹·h⁻¹, for example 3000 mL·g⁻¹·h⁻¹; in some embodiments, a molar ratio of carbon dioxide to hydrogen is 1:(1-8), for example 1:3; in some embodiments, a molar ratio of carbon dioxide to toluene is (1-30):2, for example 16:2.

In some embodiments of the present disclosure, carbon dioxide is fed at a space velocity of 300-6000 mL·g⁻¹·h⁻¹, for example 3000 mL·g⁻¹·h⁻¹; in some embodiments, hydrogen is fed at a space velocity of 900-18000 mL·g⁻¹·h⁻¹, for example 9000 mL·g⁻¹·h⁻¹; in some embodiments, toluene is fed in the form of gas, and gaseous toluene is fed at a space velocity of 25-500 mL·g⁻¹·h⁻¹, for example 250 mL·g⁻¹·h⁻¹. In some embodiments of the present disclosure, toluene is introduced by a bubbling method or a high-pressure injection pump; under the condition that toluene is introduced by a bubbling method, the reaction pressure and the temperature of the bubbling tank can be adjusted, and thus the toluene injection volume fraction can be calculated by the Antoine equation; under the condition that toluene is introduced by a high-pressure injection pump, the injection rate can be directly set. In specific embodiments of the present disclosure, toluene is bubbled from a tank at 90° C.

In some embodiments of the present disclosure, before contacting the activated catalyst with carbon dioxide, hydrogen, and toluene, the activated catalyst is mixed with a quartz sand. In some embodiments of the present disclosure, the quartz sand has a particle size of 40-60 mesh; In some embodiments, a mass ratio of the activated catalyst to the quartz sand is 1:(1-8), for example 1:4. The mixing of the activated catalyst and the quartz sand is to eliminate the influence of reaction heat on the catalytic reaction.

In some embodiments of the present disclosure, the carbon dioxide hydrogenation coupled toluene alkylation reaction is carried out at a temperature of 300-460° C., for example 360° C.; in some embodiments, the carbon dioxide hydrogenation coupled toluene alkylation reaction is carried out at a pressure of 1-5 MPa, for example 3 MPa.

In the present disclosure, the carbon dioxide hydrogenation coupled toluene alkylation reaction comprises a hydrogenation reaction of carbon dioxide to methanol and an alkylation reaction of toluene with methanol. The reaction equation of the hydrogenation reaction of carbon dioxide to methanol is as follows:

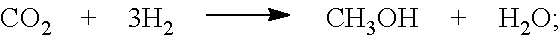

the reaction equation of the alkylation reaction of toluene with methanol is as follows:

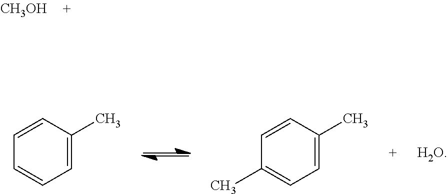

The technical solutions of the present disclosure will be clearly and completely described below in reference to the examples of the present disclosure. Obviously, the described examples are only a part of the examples of the present disclosure, rather than all the examples. Based on the examples of the present disclosure, all other examples obtained by those ordinary skilled in the art without creative labor shall fall within the protection scope of the present disclosure.

### Example 1

ZnZrO_{1.7} and H-ZSM-5 zeolite (with a silica-alumina ratio of 85, purchased from Nankai University Catalyst) with a mass ratio of 1:1 were ground and mixed to be uniform, then granulated, and sieved, obtaining a combined catalyst with a particle size of 40-60 mesh.

### Example 2

2.0 g of H-ZSM-5 zeolite (with a silica-alumina ratio of 85, purchased from Nankai University Catalyst) was taken, and 2.44 mL of tetraethyl orthosilicate and 1 mL of hexane were added, then they were mixed to be uniform, and subjected to an impregnation for 4 hours. The resulting product was dried at 110° C., and then calcined in air at 500° C. for 4 hours. The steps above were repeated twice, obtaining a modified H-ZSM-5 zeolite.

ZnZrO_{1.7} and the modified H-ZSM-5 zeolite (with a mass ratio of 1:1) were ground and mixed to be uniform, then granulated, and sieved, obtaining a combined catalyst with a particle size of 40-60 mesh.

### Example 3

ZnZrO_{1.7} and the modified H-ZSM-5 zeolite (with a mass ratio of 1:9) were ground and mixed to be uniform, then granulated, and sieved, obtaining a combined catalyst with a particle size of 40-60 mesh; wherein the modified H-ZSM-5 zeolite was prepared by the same method as described in Example 2.

### Example 4

ZnZrO_{1.7} and the modified H-ZSM-5 zeolite (with a mass ratio of 1:9) were ground and mixed to be uniform, then granulated and sieved, obtaining a combined catalyst with a particle size of 40-60 mesh; wherein the modified H-ZSM-5 zeolite was prepared by the same method as described in Example 2 except that the modification steps were repeated for 4 times rather than twice.

### Example 5

0.0164 g of zinc nitrate hexahydrate and 0.1586 g of zirconium nitrate pentahydrate were dissolved in 2.7 mL of water, labeled as Solution A; 0.0837 g of ammonium carbonate was dissolved in 2.7 mL of water, labeled as Solution B; 0.45 g of modified zeolite (the same as described in Example 2) was added to Solution A, then Solution B was added dropwise therein at 70° C. while stirring, at the speed of 3 mL·min⁻¹. After the dropping was completed, the resulting mixture was continuously stirred for another 2 hours, then cooled to room temperature, and centrifuged, obtaining a precipitate. The precipitate was in sequence washed and dried overnight, then calcined at 500° C. in air for 3 hours, granulated, and sieved, obtaining a combined catalyst with a particle size of 40-60 mesh.

### Use Example

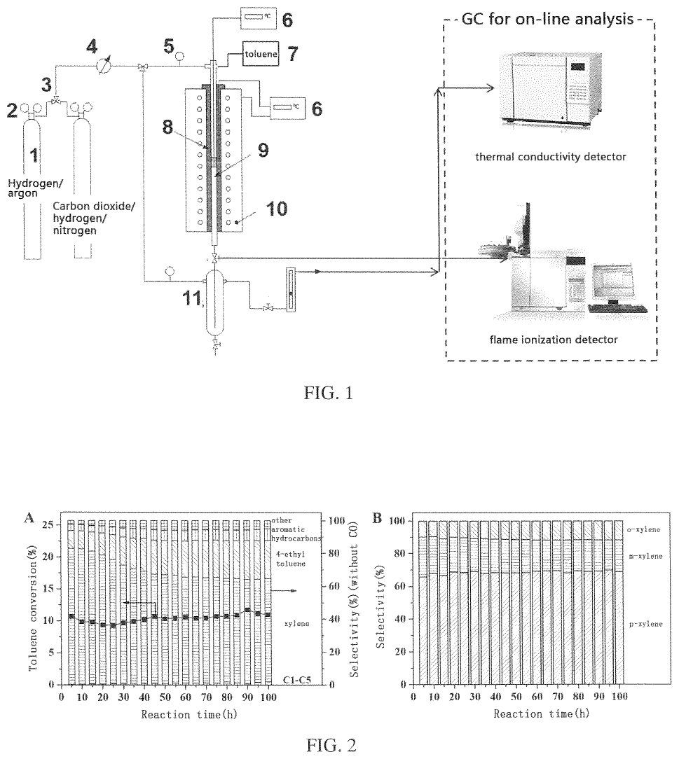

A high-pressure continuous fixed-bed reactor was used to evaluate the performance of the catalysts, and gas chromatography was used to analyze components of the products. The schematic diagram of the instrument was shown in FIG. 1. 0.2 g of the combined catalysts provided in Examples 1-5 were respectively mixed with 0.8 g of quartz sand with a particle size of 40-60 mesh to be uniform. The samples were first reduced in a hydrogen-nitrogen mixture at 450° C. for 2 h, obtaining an activated catalyst; wherein hydrogen accounts for 5% of the hydrogen-nitrogen mixture; the activated catalysts were loaded into a quartz reaction tube, and a mixture of carbon dioxide, hydrogen, toluene, and nitrogen were fed at a space velocity of 12000 mL·g⁻¹·h⁻¹ (the volume fraction of nitrogen was 1-10%, as the internal standard), in which a molar ratio of hydrogen to carbon dioxide was 3:1, a molar ratio of carbon dioxide to gaseous toluene was 12:1, and the gaseous toluene was bubbled from a tank at 90° C. The reaction was conducted at 360° C. and 3.0 MPa for 15 hours. All pipes of the instrument were equipped with heating and insulation designs, and before the reaction the temperature of the pipes was higher than that of the bubbling tank. The reaction products were analyzed by two on-line gas chromatographs (GC) equipped with the flame ionization detector (FID) and the thermal conductivity detector (TCD), respectively. The carbon dioxide conversion and reaction product selectivity were calculated by the C-based normalized method, and the results were shown in Table 1.

### Comparative Example

Only 0.18 g of the modified H-ZSM-5 zeolite as prepared in Example 2 was used as the catalyst, and the reaction was performed with the same method as described in the use example, expect that nitrogen rather than carbon dioxide and hydrogen was fed. The test results of carbon dioxide conversion and reaction products selectivity were shown in Table 1.

TABLE 1
Test results of carbon dioxide conversion and reaction products selectivity

| | | Xylene | | | | |
|---|---|---|---|---|---|---|
| | | selectivity | Xylene | | | |
| | Toluene | (%) | o-xylene | m-xylene | p-xylene | CO |
| | conversion | (without | Selectivity | Selectivity | Selectivity | Selectivity |
| | (%) | CO) | (%) | (%) | (%) | (%) |
| Example 1 | 30.5 | 77.7 | 22.1 | 52.8 | 25.1 | 8.7 |
| Example 2 | 25.5 | 66.3 | 24.5 | 26.5 | 49.0 | 8.8 |
| Example 3 | 13.9 | 92.4 | 17.6 | 48.5 | 33.9 | 0.7 |
| Example 4 | 9.8 | 81.1 | 8.9 | 20.3 | 70.8 | 1.7 |
| Example 5 | 9.8 | 87.6 | 18.6 | 44.2 | 37.2 | 8.2 |
| Comparative | 0.5 | 53.8 | 18.4 | 45.8 | 35.8 | 0 |
| Example | | | | | | |

It can be seen from Table 1 that the modified H-ZSM-5 zeolite could improve PX selectivity; adjusting the ratio of the metal oxide to the zeolite could effectively improve xylene selectivity and suppress the reversed water-gas shift (RWGS) reaction; from comparison between Example 4 and Example 2, it can be known that adjusting modification process of the zeolite could further improve PX selectivity; as can be known from the experimental results of the Comparative Example, under the reaction conditions of the Comparative Example, the toluene disproportionation reaction rate over the zeolite was quite low, and the toluene conversion was only 0.5%, and the alkylation reaction of toluene was preferred in the presence of carbon dioxide and hydrogen, indicating that xylene in Examples 1-5 was substantially all generated by the alkylation reaction of toluene with carbon dioxide and hydrogen, rather than the toluene disproportionation reaction, wherein carbon dioxide and hydrogen acted as effective alkylation reagents.

The 100-hour stability test result of the combined catalyst as prepared in Example 4 was shown in FIG. 2, where (A) in FIG. 2 shows the distribution diagram of the catalytic reaction products (without CO), and (B) in FIG. 2 was a graph illustrating the distribution of o-xylene, m-xylene, and PX in xylene. It can be seen from FIG. 2 that the conversion of toluene and the selectivity to PX in xylene were maintained at 11% and 70%, respectively. The xylene selectivity (without CO) decreased from 82% (initially) to 63% (after 30 hours), then tended to be constant, and the 4-ethyltoluene selectivity, which also has high additional value, rose from 11% to 23%. 4-ethyltoluene was formed by a further side chain alkylation of PX. This showed that the combined catalyst provided by the present disclosure had stable activity and PX selectivity, with a gaseous alkane selectivity of less than 1.5%, and obtained products always contain most of aromatic hydrocarbon components with high additional value.

The description of the above embodiments is only used to help understand the method and core idea of the present disclosure. It should be pointed out that for those ordinary skilled in the art, without departing from the principle of the present disclosure, several improvements and modifications can be made to the present disclosure, and these improvements and modifications also fall within the protection scope of the claims of the present disclosure. Various modifications to these embodiments are obvious to those skilled in the art, and the general principles defined herein can be implemented in other embodiments without departing from the spirit or scope of the present disclosure. Therefore, the present disclosure will not be limited to the embodiments shown in the text, but should conform to the widest scope consistent with the principles and novel features disclosed in the text.

What is claimed is:

1. A combined catalyst, comprising a metal oxide and a zeolite; wherein the metal oxide includes one or more of ZnZrOₓ₁, ZnCrOₓ₂, ZnAlOₓ₃, and CrOₓ₄, wherein 1<x1<2, wherein 1<x2<1.5, wherein 1<x3<1.5, and wherein 1<x4<1.5.

2. The combined catalyst as claimed in claim 1, wherein the zeolite includes one or more of ZSM-5 zeolite, MCM-22 zeolite, or SAPO-34 zeolite.

3. The combined catalyst as claimed in claim 1, wherein a mass ratio of the metal oxide to the zeolite is (1-9):(1-9).

4. A method for preparing the combined catalyst as claimed in claim 1, comprising:
mixing the metal oxide with the zeolite, to obtain the combined catalyst.

5. A method for preparing the combined catalyst as claimed in claim 1, comprising:
subjecting a metal salt solution and the zeolite to a first mixing, to obtain a dispersion liquid;
subjecting the dispersion liquid and a precipitant to a second mixing, and subjecting the resulting mixture to a precipitation reaction, to obtain a precipitate; and
calcining the precipitate, to obtain the combined catalyst.

6. A method for preparing xylene using the combined catalyst according to claim 1 by coupling carbon dioxide hydrogenation with toluene alkylation, comprising:
placing the combined catalyst in a reducing atmosphere, subjecting the combined catalyst to an activation, to obtain an activated catalyst;
contacting the activated catalyst with carbon dioxide, hydrogen and toluene, and
subjecting the activated catalyst contacted with carbon dioxide, hydrogen and toluene to a carbon dioxide hydrogenation coupled with toluene alkylation reaction, to obtain xylene.

7. The method as claimed in claim 6, wherein a gas for providing the reducing atmosphere is a mixture of hydrogen and argon, or a mixture of hydrogen and nitrogen.

8. The method as claimed in claim 6, wherein the activation is performed at a temperature of 200-600° C. for 0.5-12 hours.

9. The method as claimed in claim 6, wherein the carbon dioxide is fed at a space velocity of 300-6000 mL·g⁻¹·h⁻¹; wherein a molar ratio of carbon dioxide to hydrogen is 1:(1-8); and wherein a molar ratio of carbon dioxide to toluene is (1-30):2.

10. The method as claimed in claim 6, wherein the carbon dioxide hydrogenation coupled with toluene alkylation reaction is carried out at a temperature of 300-460° C. and a pressure of 1-5 MPa.

11. The method according to claim 5, wherein the metal salt solution is one or more of a metal nitrate solution, metal acetate solution or metal sulfate solution.

12. The method according to claim 5, wherein the concentration of the metal salt solution is 0.010 to 0.200 g·mL⁻¹.

13. The method according to claim 5, wherein the precipitant is one or more of ammonia, ammonium carbonate, sodium hydroxide, potassium hydroxide, sodium carbonate, potassium carbonate, sodium bicarbonate or potassium bicarbonate.
